(19) United States
(12) Patent Application Publication
(10) Pub. No.: US 2004/0192664 A1
(43) Pub. Date: Sep. 30, 2004

(54) Copper-carbene complexes and their use

(76) Inventors: Klaus Kunz, Dusseldorf (DE); Ulrich Scholz, Mulheim (DE); Oliver Gaertzen, Koln (DE); Dirk Ganzer, Leverkusen (DE); Joachim Wesener, Koln (DE)

Correspondence Address: LANXESS CORPORATION, PATENT DEPARTMENT/ BLDG 14, 100 BAYER ROAD, PITTSBURGH, PA, 15205-9741, US

(21) Appl. No.: 10/752,353

(22) Filed: Jan. 6, 2004

(30) Foreign Application Priority Data
Jan. 7, 2003 (DE) 10300098.4

(51) Int. Cl. A61K 31/555; C07F 1/08

(52) U.S. Cl. 514/184; 548/101

(57) ABSTRACT

The invention relates to copper-carbene complexes, to a process for preparing them and to their use in catalytic coupling reactions.

### BACKGROUND OF THE INVENTION

1. Field of the Invention

The invention relates to copper-carbene complexes, to a process for preparing them and to their use in catalytic coupling reactions.

2. Brief Description of the Prior Art

Of interest here is the formation of carbon-heteroatom bonds which have gained enormous importance in modern synthesis. For example, N-substituted anilines are prepared, for example, by coupling activated chloro-, bromo- or iodoaromatics with primary or secondary amines in the presence of a palladium catalyst, of a phosphine and of a base (see Hartwig, Angew. Chem., Int. Ed. 1998, 37, 2046-2067; Buchwald, Top. Curr. Chem. 2002, 219, 131-209). Alternatively, instead of the phosphines, N-heterocyclic carbenes can also be used (WO 01/66248).

The disadvantage of the syntheses described above is the use of palladium, which is expensive, and subject to severe price fluctuations, and only recyclable with difficulty.

Alternatively, Venkataraman et al. (Tetrahedron Letters, 2001, 42, 4791-4793) disclose the use of preformed complexes of copper dibromide and triphenylphosphine for use in the addition of aryl halides to secondary aromatic amines. However, a disadvantage of this method is the often low chemoselectivity and the narrow spectrum of reactions in which technically acceptable conversions and conversion rates can be achieved. In addition, the oxidation sensitivity of phosphines is problematic.

There is therefore the need to provide catalysts which are simple to prepare and afford the desired products in good yields in coupling reactions.

### SUMMARY OF THE INVENTION

The present invention therefore provides copper complexes containing ligands of the formula (I)

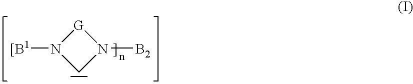

in which

G is a 1,2-ethanediyl or 1,2-ethenediyl radical which is optionally mono- or polysubstituted and

B¹ is C₅-C₁₈-aryl, C₁-C₁₈-alkyl which may optionally have one or more heteroatoms from the group of oxygen, nitrogen or sulphur, or C₆-C₁₉-aralkyl and

B² is an n-valent radical having a total of 2 to 40 carbon atoms and

n is 1, 2 or 3.

In the context of the invention, all radical definitions, parameters and illustrations hereinabove and listed hereinbelow, in general or within areas of preference, i.e. the particular areas and areas of preference too, may be combined as desired.

### DETAILED DESCRIPTION OF THE INVENTION

Alkyl, alkoxy, alkylene and alkenylene are in each case independently a straight-chain, cyclic, branched or unbranched alkyl, alkoxy, alkylene and alkenylene radical respectively, which may optionally be further substituted by C₁-C₄-alkoxy. The same applies to the nonaromatic moiety of an arylalkyl radical.

C₁-C₄-Alkyl is, for example, methyl, ethyl, n-propyl, isopropyl, n-butyl, sec-butyl and tert-butyl; C₁-C₈-alkyl is additionally, for example, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 1-ethyl-2-methylpropyl, n-heptyl and n-octyl; C₁-C₁₂-alkyl is further additionally, for example, adamantyl, the isomeric menthyls, n-nonyl, n-decyl and n-dodecyl, and C₁-C₁₈-alkyl is still further additionally, for example, n-octadecyl.

C₁-C₄-Alkoxy is, for example, methoxy, ethoxy, n-propoxy, isopropoxy, n-butoxy, sec-butoxy and tert-butoxy; C₁-C₈-alkoxy is additionally n-pentoxy, 1-methylbutoxy, 2-methylbutoxy, 3-methylbutoxy, neopentoxy, 1-ethylpropoxy, cyclohexoxy, cyclo-pentoxy, n-hexoxy and n-octoxy, and C₁-C₁₂-alkoxy is further additionally, for example, adamantoxy, the isomeric menthoxy radicals, n-decoxy and n-dodecoxy.

C₁-C₈-Alkylene is, for example, methylene, 1,1-ethylene, 1,2-ethylene, 1,1-propylene, 1,3-propylene, 1,4-butylene, 1,2-cyclohexoxylene and 1,2-cyclo-pentylene.

Haloalkyl is in each case independently a straight-chain, cyclic, branched or unbranched alkyl radical which is singly, multiply or fully substituted by chlorine or fluorine atoms.

For example, C₁-C₈-haloalkyl is trifluoromethyl, chlorodifluoromethyl, 2,2,2-trifluoroethyl, pentafluoroethyl, nonafluorobutyl, heptafluoroisopropyl and perfluorooctyl.

Aryl is in each case independently a heteroaromatic radical having 5 to 18 framework carbon atoms of which no, one, two or three framework carbon atoms per cycle, but at least one framework carbon atom in the entire molecule, may be substituted by heteroatoms selected from the group of nitrogen, sulphur or oxygen, but is preferably a carbocyclic aromatic radical having 6 to 18 framework carbon atoms.

Examples of carbocyclic aromatic radicals having 6 to 18 framework carbon atoms are phenyl, naphthyl, phenanthrenyl, anthracenyl or fluorenyl; heteroaromatic radicals having 5 to 14 framework carbon atoms of which no, one, two or three framework carbon atoms per cycle, but at least one framework carbon atom in the entire molecule, may be substituted by heteroatoms selected from the group of nitrogen, sulphur or oxygen are, for example, pyridinyl, oxazolyl, imidazolyl, benzofuranyl, dibenzofuranyl or quinolinyl.

In addition, the carbocyclic aromatic radical or heteroaromatic radical may be substituted by up to five identical or different substituents per cycle which are selected from the group of free or protected hydroxyl, cyano, chlorine, fluorine, C₁-C₁₂-alkyl, C₁-C₁₂-haloalkyl, CO(C₁-C₁₂-alkyl), COO(C₁-C₁₂-alkyl), CO(C₅-C₁₈-aryl), COO(C₅-C₁₈-aryl), CON(C₁-C₁₂-alkyl)₂, C₅-C₁₈-aryl, C₁-C₁₂-alkoxy, C₁-C₁₂-haloalkoxy, di(C₁-C₈-alkyl)amino or tri(C₁-C₈-alkyl)siloxyl.

Arylalkyl is in each case independently a straight-chain, cyclic, branched or unbranched alkyl radical as defined above which may be singly, multiply or fully substituted by aryl radicals as defined above.

The preferred substitution patterns are defined hereinbelow:

G is preferably a 1,2-ethanediyl or 1,2-ethenediyl radical which is optionally mono- or polysubstituted by C₁-C₈-alkyl, more preferably a 1,2-ethenediyl radical.

B¹, in the case that n=1, is preferably C₅-C₁₈-heteroaryl or C₁-C₁₈-alkyl, each of which contains one or more heteroatoms from the group of oxygen, nitrogen or sulphur, and, in the case that n=2 or 3, is preferably C₁-C₁₈-alkyl, C₆-C₁₁-aralkyl or C₆-C₁₀-aryl.

B¹, in the case that n=1, is more preferably pyridinyl, oxazolyl, imidazolylalkyl, benzofuranyl, furanyl, quinolinyl, piperidinyl, pyrrolidinyl, tetrahydrothiophenyl or tetrahydrofuranyl, and the radicals mentioned may optionally be substituted by C₁-C₁₂-alkyl, C₁-C₁₂-haloalkyl or C₁-C₈-alkoxy, or in the case that n=2.

B², in the case that n=1, is preferably C₅-C₁₈-heteroaryl or C₁-C₁₈-alkyl, each of which contains one or more heteroatoms from the group of oxygen, nitrogen or sulphur, and, in the case that n=2 or 3, is preferably a divalent radical from the group of (C₁-C₈-alkylene)-(C₅-C₁₉-aryl)-(alkylene-C₁-C₈), (C₁-C₈-alkylene)-(C₅-C₁₉-arylene), C₂-C₈-alkylene, C₂-C₈-alkenylene, C₅-C₁₉-arylene, C₁₀-C₃₈-bisarylene and C₄-C₁₈-alkylene, each of which contains one or more heteroatoms from the group of oxygen, nitrogen or sulphur.

B², in the case that n=1, is more preferably pyridinyl, oxazolyl, imidazolylalkyl, benzofuranyl, furanyl, quinolinyl, piperidinyl, pyrrolidinyl, tetrahydrothiophenyl or tetrahydrofuranyl, and the radicals mentioned may optionally be substituted by C₁-C₁₂-alkyl, C₁-C₁₂-haloalkyl or C₁-C₈-alkoxy, or, in the case that n=2, is more preferably a divalent radical from the group of 1,1-(C₁-C₄-alkylene)-(C₅-C₁₉-arylene)-1,1-(alkylene-C₁-C₄), 1,1-(C₁-C₄-alkylene)-(piperidinediyl)-1,1-(alkylene-C₁-C₄), 1,1-(C₁-C₄-alkylene)-(pyrrolidinediyl)-1,1-(alkylene-C₁-C₄), 1,1-(C₁-C₄-alkylene)-(C₅-C₁g-arylene), C₂-C₄-alkylene, C₂-C₄-alkenylene, C₅-C₁₁-arylene or C₁₀-C₂₂-bisarylene.

B², in the case that n=1, is even more preferably 2-pyridinyl, 2-piperidinylalkyl, 2-pyrrolidinyl, 2-tetrahydrothiophenyl or 2-tetrahydrofuranyl, and the radicals mentioned may optionally be substituted by C₁-C₄-alkyl, and is still more preferably 2-pyridinyl, or, in the case that n=2, is more preferably a divalent radical from the group of 2,6-di-1,1-(C₁-C₄-alkylene)pyridine and 2,6-di-1,1-(C₁-C₄-alkylene)benzene, and the radicals mentioned may optionally be further substituted by C₁-C₄-alkyl radicals.

n is preferably 1 or 2.

Particularly preferred copper complexes containing compounds of the formula (I) are those of the formula (Ia),

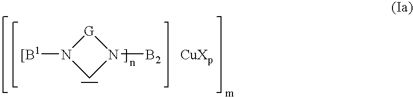

in which

n, G, B¹ und B² have the definitions and areas of preference specified above and

X is halide, (C₁-C₈-haloalkyl)carboxylate, (C₁-C₈-alkyl)carboxylate, (C₁-C₈-haloalkyl)sulphonate, (C₅-C₁₈-aryl)sulphonate, cyanide, optionally fluorinated acetylacetonate, nitrate, oxinate, phosphate, carbonate, hexafluorophosphate, tetraphenylborate, tetrakis(pentafluorophenyl)borate or tetrafluoroborate, preferably chloride, bromide, iodide, trifluoroacetate, acetate, propionate, methanesulphonate, trifluoromethanesulphonate, nonafluorobutanesulphonate, tosylate, acetylacetonate, nitrate, hexafluorophospate or tetrafluoroborate, and more preferably chloride, bromide, iodide, and

p is 0, 1 or 2, preferably 2, and

m is 1, 2, 3, 4, 5 or 6, preferably 1 or 2.

The copper complexes according to the invention may in some cases also occur in the form of salt adducts which are of course likewise encompassed by the invention.

Very particularly preferred copper complexes containing ligands of the formula (I) are:

[(N,N-dipyridyl-imidazolylidene)copper dibromide], [2,6-{bis-N-(N-methylimidazolylidene)methyl)pyridine}copper dibromide] and [1,3-{bis-N-(N-methylimidazolylidene)methyl)-5-methylbenzene}copper dibromide].

The inventive copper complexes containing ligands of the formula (I) can be prepared, for example, by reacting compounds of the formula (II)

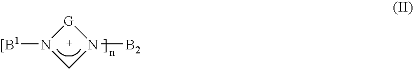

in which

n, G, B¹ and B² each have the definitions and areas of preference specified under the formula (I) with compounds of the formula (III)

Cu—Xₚ (III)

in which

X and p each have the definitions and areas of preference specified in the formula (Ia),

the conversion taking place in the presence of or after reaction of compounds of the formula (II) with base.

Useful bases are, for example, alkaline earth metal or alkali metal hydrides, hydroxides, amides and/or alkoxides, and also organolithium compounds.

In cases in which p=0, it is also possible, for example, to use copper powder. Preferred compounds of the formula (III) are:

copper(I) oxide, copper(II) oxide, copper(I) chloride, copper(I) bromide, copper(I) iodide, copper(II) bromide, copper(II) chloride, copper(I) trifluoromethanesulphonate, copper(II) acetate, copper(II) acetylacetonate or mixtures thereof.

The molar ratio of compounds of the formula (II) to copper atoms in compounds of the formula (III) in the preparation of copper complexes containing ligands of the formula (I) may generally be 3:1 to 0.5:1, preferably 2:1 to 1:1, more preferably 1.2:1 to 1:1.

The copper complexes containing ligands of the formula (I) can be prepared separately in an inert organic solvent suitable for this purpose, for example tetrahydrofuran, diethyl ether, toluene, xylene, chloroform, dichloromethane, methanol and/or ethanol.

The amount of solvent to be used can be determined by appropriate preliminary experiments.

The copper complexes containing ligands of the formula (I) are then prepared from the starting compounds of the formulae (II) and (III) described, for example, by admixing compounds of the formula (II) with base and adding compounds of the formula (III).

The inventive copper complexes containing ligands of the formula (I) are suitable in particular for catalytically forming carbon-nitrogen, carbon-oxygen and carbon-sulphur bonds, and also for preparing alkines.

The invention therefore also encompasses catalysts which comprise the copper complexes according to the invention.

In addition, the invention also encompasses a process for preparing compounds of the formula (IV),

Ar—(F—R²)ₙ (IV)

in which

n is 1, 2 or 3 and

Ar is a substituted or unsubstituted aromatic radical and

F is oxygen, sulphur, NR³, NR³CO or ethinediyl, where R³ is hydrogen, C₁-C₁₂-alkyl, C₅-C₁₈-aryl or C₆-C₁₉-arylalkyl and

R² is Ar, C₁-C₁₂-alkyl, C₁-C₁₂-haloalkyl, C₂-C₁₂-alkenyl or C₆-C₁₉-arylalkyl,

which is characterized in that compounds of the formula (V)

Ar—Z (V)

in which

Ar is as defined above and

Z is chlorine, bromine, iodine, a diazonium salt or a sulphonate

are reacted with compounds of the formula (VI)

H—F—R² (VI)

in which

F and R² are each as defined above and

the conversion is effected in the presence of base and copper complexes containing ligands of the formula (I).

The areas of preference for compounds of the formulae (IV) to (VI) are defined hereinbelow:

Ar is preferably a carbocyclic aromatic radical having 6 to 24 framework carbon atoms or a heteroaromatic radical having 5 to 24 framework atoms, of which no, one, two or three framework atoms per cycle, but at least one framework atom in the entire molecule, are heteroatoms which are selected from the group of nitrogen, sulphur or oxygen. The carbocyclic aromatic radicals or the heteroaromatic radicals may also be substituted by up to five identical or different substituents per cycle which are selected from the group of hydroxyl, chlorine, fluorine, nitro, cyano, free or protected formyl, C₁-C₁₂-alkyl, C₅-C₁₄-aryl, C₆-C₁₅-arylalkyl, —PO—[(C₁-C₈)alkyl]₂, —PO—[(C₅-C₁₄)aryl]₂, —PO—[(C₁-C₈)alkyl)(C₅-C₁₄)aryl)]tri(C₁-C₈-alkyl)siloxyl or radicals of the formula (VIIa-f),

| | | | |
|---|---|---|---|
| A-B-D-E | (VIIa) | A-E | (VIIb) |
| A-SO₂-E | (VIIc) | A-B-SO₂R⁴ | (VIId) |
| A-SO₃W | (VIIe) | A-COW | (VIIf) |

in which, each independently,

A is absent or is a C₁-C₈-alkylene radical and

B is absent or is oxygen, sulphur or NR⁴,

where R⁴ is hydrogen, C₁-C₈-alkyl, C₆-C₁₅-arylalkyl or C₅-C₁₄-aryl and

D is a carbonyl group and

E is R⁵, OR⁵, NHR⁶ or N(R⁶)₂,

where

R⁵ is C₁-C₈-alkyl, C₆-C₁₅-arylalkyl, C₁-C₈-haloalkyl or C₅-C₁₄-aryl and

R⁶ is in each case independently C₁-C₈-alkyl, C₆-C₁₅-arylalkyl or C₅-C₁₄-aryl, or N(R⁶)₂ together is a cyclic amino radical and

W is OH, NH₂, or OM where M may be an alkali metal ion, half an equivalent of an alkaline earth metal ion, an ammonium ion or an organic ammonium ion.

Ar is more preferably phenyl, naphthyl, phenanthrenyl, anthracenyl, biphenyl, binaphthyl, fluorenyl, pyridinyl, oxazolyl, thiophenyl, benzofuranyl, benzothiophenyl, dibenzofuranyl, dibenzothiophenyl, furanyl, indolyl, pyridazinyl, pyrazinyl, pyrimidinyl, triazolyl and quinolinyl, and the radicals mentioned may additionally be further substituted by no, one, two or three radicals per cycle, each of which is selected independently from the group of fluorine, nitro, cyano, di(C₁-C₄-alkyl)amino, C₁-C₄-alkyl, C₅-C₁₀-aryl, C₁-C₈-fluoroalkyl, C₁-C₈-fluoroalkoxy, C₁-C₈-alkoxy, CO(C₁-C₄-alkyl), COO-(C₁-C₄)-alkyl, —CON(C₁-C₄-alkyl)₂.

Ar is even more preferably a phenyl radical which may be further substituted by no, one, two or three radicals, each of which is selected independently from the group of nitro, fluorine, cyano, C₁-C₄-alkyl, C₁-C₄-alkoxy, trifluoromethyl, trifluoromethoxy, CO-(C₁-C₄)-alkyl, COO-(C₁-C₄)-alkyl and —CON(C₁-C₄-alkyl)₂.

n is preferably 1.

Z is preferably chlorine, bromine or iodine.

F is preferably oxygen, sulphur, NR³ or ethinediyl, where R³ is hydrogen or C₁-C₄-alkyl.

R² is preferably Ar or C₁-C₁₂-alkyl.

For the process according to the invention, the copper complexes containing ligands of the formula (I) are generally used in amounts of 0.02 mol % to 10 mol %, preferably 0.1 mol % to 3 mol %, based on the compounds of the formula (IV) used.

Useful bases in the process according to the invention are, for example and with preference, alkali metal and/or alkaline earth metal carbonates, hydrogencarbonates, alkoxides, phosphates, fluorides and/or hydroxides, and particular mention should be made of potassium carbonate and/or sodium carbonate, caesium carbonate, caesium hydrogencarbonate, sodium methoxide, potassium tert-butoxide, potassium amylate, caesium fluoride, potassium phosphate and barium hydroxide. Preference is given to using potassium carbonate, sodium carbonate, caesium carbonate and/or caesium hydrogencarbonate.

Per mole of HaI in compounds of the formula (IV) to be exchanged, for example, 0.05 to 10 mol of base can be used, preferably 0.3 to 2 mol.

It is advantageous for the process according to the invention when the bases used are pretreated by grinding and/or drying.

After the grinding, the specific surface areas of the bases are preferably from approx. 0.1 to 10 m²/ g, more preferably from 0.2 to 1 m²/g (BET).

As a consequence of the marked hygroscopic properties of the bases used in the process according to the invention, the phosphates and carbonates in particular tend to absorb atmospheric constituents such as water and carbon dioxide to a greater or lesser extent. From an absorption of approx. 30 per cent by weight of atmospheric constituents, a distinct influence on the conversions to be attained can be detected. Therefore, drying of the bases in addition to the grinding is often appropriate.

Depending on the nature of the base used, the bases are dried, for example, by heating to temperatures of approx. 50 to 200° C., preferably 100 to 160° C., under a reduced pressure of approx. 0.01 to 100 mbar for several hours.

The molar ratio of compounds of the formula (VI) to compounds of the formula (IV) may be, for example, 0.8 to 10, preferably 1 to 6 and more preferably 1.1 to 4.

The process according to the invention can be carried out, for example, at temperatures of 20 to 250° C., preferably at 100 to 200° C. The optimum reaction temperatures depend on the type of the starting products, of the catalyst and of the bases used and can be determined by simple preliminary experiments.

The process according to the invention can be carried out either in the presence or in the absence of a suitable solvent. Useful solvents are, for example, aliphatic, alicyclic or aromatic hydrocarbons, for example benzine, benzene, toluene, xylene, petroleum ether, hexane, cyclohexane; ethers such as diethyl ether, diisopropyl ether, dioxane, tetrahydrofuran or ethylene glycol dimethyl ether or ethylene glycol diethyl ether; amides, for example N,N-dimethylformamide, N,N-dimethylacetamide, N-methyl-formanilide, N-methylpyrrolidone or hexamethylphosphoramide; esters such as methyl acetate or ethyl acetate, or mixtures of such solvents.

In some cases, an excess of compounds of the formula (VI) can also serve as the reaction medium.

An azeotropic agent can optionally be added to the process according to the invention and continuously removes any water formed during the reaction azeotropically in the distillation.

The process according to the invention can be carried out by customary methods in continuous or batchwise mode.

The advantage of the present invention is in particular the simple preparation of the copper complexes containing ligands of the formula (I) and the high efficiency with which the copper complexes according to the invention can be used to prepare compounds of the formula (VI).

The invention is further described by the following illustrative but non-limiting examples.

### EXAMPLES

### Preparation of Copper Complexes Containing Ligands of the Formula (I)

### Example 1

### Preparation of [1,3-{bis-N-(N-methylimidazolylidene)methyl)-5-methylbenzene}copper dibromide].2 KBr

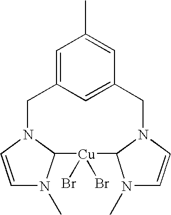

Under an argon atmosphere, 1,3-{bis-N-(N-methylimidazolium)methyl)-5-methylbenzene dichloride (135 mg, 0.31 mmol) is dissolved in 10 ml of toluene and admixed at 0° C. with potassium tert-butoxide (71 mg, 0.63 mmol). After 2 h, copper(II) bromide (70 mg, 0.31 mmol) is added and the mixture is stirred for a further 12 h. Subsequently, the solvent is removed under reduced pressure and the product is obtained as a light powder.

FD-MS: 343 (M−2Br, main component), 423 (M−Br), 503 (M+2H)

### Example 2

### Preparation of [2,6-{bis-N-(N-methylimidazolylidene)methyl)pyridine}copper dibromide].2 KBr

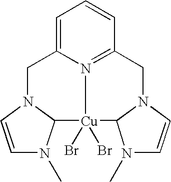

Under an argon atmosphere, 2,6-{bis-N-(N-methylimidazolium)methyl)pyridinium trichloride (131 mg, 0.31 mmol) is dissolved in 10 ml of toluene and admixed at 0° C. with potassium tert-butoxide (71 mg, 0.63 mmol). After 2 h, copper(II) bromide (70 mg, 0.31 mmol) is added and the mixture is stirred for a further 12 h. Subsequently, the solvent is removed under reduced pressure and the product is obtained as a light powder.

FD-MS: 330 (M−2Br, main component), 410 (M−Br), 490 (M+2H)

### Example 3

### Preparation of [(N,N-dipyridyl-imidazolylidene)copper dibromide].2 KBr

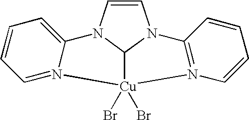

Under an argon atmosphere, [(N,N-dipyridyl-imidazolium) chloride (92 mg, 0.31 mmol) is dissolved in 10 ml of toluene and admixed at 0° C. with potassium tert-butoxide (36 mg, 0.31 mmol). After 2 h, copper(II) bromide (70 mg, 0.31 mmol) is added and the mixture is stirred for a further 12 h. Subsequently, the solvent is removed under reduced pressure and the product is obtained as a light powder.

FD-MS: 364 (M−Br, main component).

### Examples 4 to 30

### Couplings using the catalysts from Examples 1 to 3

### Example 4

### Coupling of 3-iodotrifluoromethylbenzene with n-octanethiol (catalyst from Example 1)

1.8 g (6.7 mmol) of 3-iodotrifluoromethylbenzene, 1.0 g (6.7 mmol) of n-octanethiol, 1.4 g (10.1 mmol) of caesium carbonate and 500 mg (0.7 mmol) of the catalyst from Example 1 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 390 mg (20%) of product are obtained.

GC-MS/EI: 290 (M)

### Example 5

### Coupling of p-bromoacetophenone with n-octanethiol (catalyst from Example 1)

1.35 g (6.7 mmol) of p-bromoacetophenone, 1.0 g (6.7 mmol) of n-octanethiol, 1.4 g (10.1 mmol) of caesium carbonate and 500 mg (0.7 mmol) of the catalyst from Example 1 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 800 mg (45%) of product are obtained.

GC-MS/EI: 264 (M)

### Example 6

### Coupling of 3-iodotrifluoromethylbenzene with thiophenol (catalyst from Example 1)

3.6 g (13.5 mmol) of 3-iodotrifluoromethylbenzene, 1.5 g (13.5 mmol) of thiophenol, 2.8 g (20.2 mmol) of caesium carbonate and 1.0 g (1.4 mmol) of the catalyst from Example 1 are stirred in 100 ml of dioxane under an argon atmosphere at 110° C. for 12 h. The reaction solution is subsequently admixed with 50 ml of aqueous ammonia and extracted with ethyl acetate, and the combined organic extracts are dried under reduced pressure. After workup by column chromatography (hexane), 1.37 g (40%) of product are obtained.

GC-MS/EI: 254 (M)

### Example 7

### Coupling of p-bromoacetophenone with thiophenol (catalyst from Example 1)

1.3 g (6.7 mmol) of p-bromoacetophenone, 0.75 g (6.7 mmol) of thiophenol, 1.8 g (13.5 mmol) of potassium carbonate and 500 mg (0.7 mmol) of the catalyst from Example 1 are stirred in 50 ml of dioxane under an argon atmosphere at 110° C. for 12 h. The reaction solution is subsequently admixed with 20 ml of aqueous ammonia and extracted with ethyl acetate, and the combined organic extracts are dried under reduced pressure. After workup by column chromatography (hexane), 840 mg (55%) of product are obtained.

GC-MS/EI: 228 (M)

### Example 8

### Coupling of p-chloronitrobenzene with thiophenol (catalyst from Example 1)

2.1 g (13.5 mmol) of p-chloronitrobenzene, 1.5 g (13.5 mmol) of thiophenol, 2.8 g (20.2 mmol) of caesium carbonate and 1.0 g (1.4 mmol) of the catalyst from Example 1 are stirred in 100 ml of dioxane under an argon atmosphere at 110° C. for 12 h. The reaction solution is subsequently admixed with 50 ml of aqueous ammonia and extracted with ethyl acetate, and the combined organic extracts are dried under reduced pressure. After workup by column chromatography (hexane), 1.77 g (57%) of product are obtained.

GC-MS/EI: 228 (M)

### Example 9

### Coupling of 3-iodotrifluoromethylbenzene with o-hydroxypyridine (catalyst from Example 1)

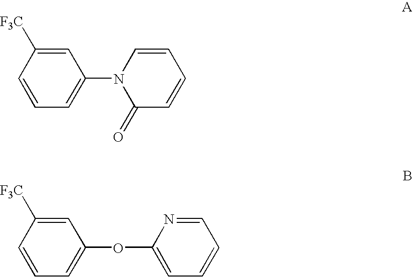

5.7 g (21 mmol) of 3-iodotrifluoromethylbenzene, 1.0 g (11 mmol) of o-hydroxypyridine, 2.9 g (21 mmol) of potassium carbonate and 1.0 g (1.4 mmol) of the catalyst from Example 1 are stirred in 100 ml of dioxane under an argon atmosphere at 110° C. for 12 h. The reaction solution is subsequently admixed with 80 ml of aqueous ammonia and extracted with ethyl acetate, and the combined organic extracts are dried under reduced pressure. After workup by column chromatography (hexane), 1.0 g (40%) of a product mixture of A and B is obtained.

CI-MS: 238 (M)

### Example 10

### Coupling of 3-iodotrifluoromethylbenzene with methanol (catalyst from Example 1)

1.8 g (6.7 mmol) of 3-iodotrifluoromethylbenzene, 2 ml (20 mmol) of ethyl acetate, 17.5 ml of a 30% sodium methoxide solution and 500 mg (0.7 mmol) of the catalyst from Example 1 are refluxed under an argon atmosphere for 12 h. The reaction solution is subsequently cautiously hydrolysed and extracted with dichloromethane, and the combined organic extracts are dried under reduced pressure. After workup by column chromatography (hexane), 1.11 g (95%) of the product were obtained.

GC-MS/EI: 176 (M)

### Example 11

### Coupling of p-bromoacetophenone with methanol (catalyst from Example 1)

2.7 g (13.5 mmol) of p-bromoacetophenone, 4 ml (40 mmol) of ethyl acetate, 35 ml of a 30% sodium methoxide solution and 1.0 g (1.4 mmol) of the catalyst from Example 1 are refluxed under an argon atmosphere for 12 h. The reaction solution is subsequently hydrolysed cautiously and extracted with dichloromethane, and the combined organic extracts are dried under reduced pressure. In addition to the product in a high fraction, GC shows fragments of the aldol by-product. After workup by column chromatography (hexane), 800 mg (40%) of the product were obtained in addition to some by-products.

GC-MS/EI: 150 (M)

### Example 12

### Coupling of p-bromotoluene with methanol (catalyst from Example 1)

2.3 g (13.5 mmol) of p-bromotoluene, 4 ml (40 mmol) of ethyl acetate, 35 ml of a 30% sodium methoxide solution and 1.0 g (1.4 mmol) of the catalyst from Example 1 are refluxed under an argon atmosphere for 12 h. The reaction solution is subsequently hydrolysed cautiously and extracted with dichloromethane, and the combined organic extracts are dried under reduced pressure. After workup by column chromatography (hexane), 1.5 g (90%) of the product were obtained.

GC-MS/EI: 122 (M)

### Example 13

### Coupling of p-chloronitrobenzene with methanol (catalyst from Example 1)

2.1 g (13.5 mmol) of p-chloronitrobenzene, 4 ml (40 mmol) of ethyl acetate, 35 ml of a 30% sodium methoxide solution and 1.0 g (1.4 mmol) of the catalyst from Example 1 are refluxed under an argon atmosphere for 12 h. The GC analysis of the crude product indicates product formation (GC-MS/EI: 153 (M)) with 70% conversion.

### Example 14

### Coupling of 3-iodotrifluoromethylbenzene with phenylacetylene (catalyst from Example 1)

2.7 g (10 mmol) of 3-iodotrifluoromethylbenzene, 1.3 g (12.5 mmol) of phenylacetylene, 2.2 g (20 mmol) of potassium tert-butoxide and 500 mg (0.7 mmol) of the catalyst from Example 1 are stirred in 100 ml of dioxane under an argon atmosphere at 110° C. for 21 h. The reaction solution is filtered and dried under reduced pressure. After workup by column chromatography (hexane), 1.92 g (78%) of product are obtained.

GC-MS/EI: 246 (M)

### Example 15

### Coupling of p-bromoacetophenone with n-octanethiol (catalyst from Example 2)

1.35 g (6.7 mmol) of p-bromoacetophenone, 1.0 g (6.7 mmol) of n-octanethiol, 1.8 g (13.5 mmol) of potassium carbonate and 490 mg (0.7 mmol) of the catalyst from Example 2 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 1.33 g (75%) of product are obtained.

### Example 16

### Coupling of p-bromoacetophenone with n-octanethiol (catalyst from Example 3)

1.35 g (6.7 mmol) of p-bromoacetophenone, 1.0 g (6.7 mmol) of n-octanethiol, 1.8 g (13.5 mmol) of potassium carbonate and 380 mg (0.7 mmol) of the catalyst from Example 3 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 880 mg (50%) of product are obtained.

### Example 17

### Coupling of p-bromoacetophenone with thiophenol (catalyst from Example 2)

1.35 g (6.7 mmol) of p-bromoacetophenone, 0.75 g (6.7 mmol) of thiophenol, 1.8 g (13.5 mmol) of potassium carbonate and 490 mg (0.7 mmol) of the catalyst from Example 2 are stirred in 50 ml of dioxane under an argon atmosphere at 110° C. for 12 h. The reaction solution is subsequently admixed with 20 ml of aqueous ammonia and extracted with ethyl acetate, and the combined organic extracts are dried under reduced pressure. After workup by column chromatography (hexane), 810 mg (54%) of product are obtained.

### Example 18

### Coupling of p-bromoacetophenone with thiophenol (catalyst from Example 3)

1.35 g (6.7 mmol) of p-bromoacetophenone, 0.75 g (6.7 mmol) of thiophenol, 1.8 g (13.5 mmol) of potassium carbonate and 380 mg (0.7 mmol) of the catalyst from Example 3 are stirred in 50 ml of dioxane under an argon atmosphere at 110° C. for 12 h. The reaction solution is subsequently admixed with 20 ml of aqueous ammonia and extracted with ethyl acetate, and the combined organic extracts are dried under reduced pressure. After workup by column chromatography (hexane), 1.3 g (85%) of product are obtained.

### Example 19

### Coupling of p-bromoacetophenone with phenol (catalyst from Example 2)

1.35 g (6.7 mmol) of p-bromoacetophenone, 630 mg (6.7 mmol) of phenol, 1.8 g (13.5 mmol) of potassium carbonate and 490 mg (0.7 mmol) of the catalyst from Example 2 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 210 mg (15%) of product are obtained.

### Example 20

### Coupling of p-bromoacetophenone with o-hydroxypyridine (catalyst from Example 3)

1.35 g (6.7 mmol) of p-bromoacetophenone, 650 mg (6.7 mmol) of o-hydroxypyridine, 1.8 g (13.5 mmol) of potassium carbonate and 380 mg (0.7 mmol) of the catalyst from Example 3 are stirred in 50 ml of dioxane under an argon atmosphere at 110° C. for 12 h. The reaction solution is subsequently admixed with 40 ml of aqueous ammonia and extracted with ethyl acetate, and the combined organic extracts are dried under reduced pressure. After workup by column chromatography (hexane), 880 mg (62%) of a product mixture of A and B (ratio 9:1) are obtained.

### Example 21

### Coupling of p-bromoacetophenone with methanol (catalyst from Example 2)

1.34 g (13.5 mmol) of p-bromoacetophenone, 2 ml (20 mmol) of ethyl acetate, 17.5 ml of a 30% sodium methoxide solution and 490 mg (0.7 mmol) of the catalyst from Example 2 are refluxed under an argon atmosphere for 12 h. The reaction solution is subsequently hydrolysed cautiously and extracted with dichloromethane, and the combined organic extracts are dried under reduced pressure. In addition to the product (35%) in a high proportion, the GC shows fragments of the aldol by-product.

### Example 22

### Coupling of p-bromoacetophenone with methanol (catalyst from Example 3)

1.34 g (13.5 mmol) of p-bromoacetophenone, 2 ml (20 mmol) of ethyl acetate, 17.5 ml of a 30% sodium methoxide solution and 380 mg (0.7 mmol) of the catalyst from Example 3 are refluxed under an argon atmosphere for 12 h. The reaction solution is subsequently hydrolysed cautiously and extracted with dichloromethane, and the combined organic extracts are dried under reduced pressure. In addition to the product (40%) in a high proportion, the GC shows fragments of the aldol by-product.

### Example 23

### Coupling of 3-iodotrifluoromethylbenzene with phenylacetylene (catalyst from Example 2)

2.7 g (10 mmol) of 3-iodotrifluoromethylbenzene, 1.3 g (12.5 mmol) of phenylacetylene, 2.2 g (20 mmol) of potassium tert-butoxide and 490 mg (0.7 mmol) of the catalyst from Example 2 are stirred in 100 ml of dioxane under an argon atmosphere at 110° C. for 21 h. The GC analysis of the crude product indicates the formation of product (cf. with GC-MS/EI GZN 276-11) with 72% conversion.

### Example 24

### Coupling of 3-iodotrifluoromethylbenzene with phenylacetylene (catalyst from Example 3)

2.7 g (10 mmol) of 3-iodotrifluoromethylbenzene, 1.3 g (12.5 mmol) of phenylacetylene, 2.2 g (20 mmol) of potassium tert-butoxide and 380 mg (0.7 mmol) of the catalyst from Example 3 are stirred in 100 ml of dioxane under an argon atmosphere at 110° C. for 21 h. The GC analysis of the crude product indicates the formation of product with 70% conversion.

### Example 25

### Coupling of bromobenzene with aniline (catalyst from Example 1)

1.05 g (6.7 mmol) of bromobenzene, 1.4 g (10.1 mmol) of caesium carbonate and 500 mg (0.7 mmol) of the catalyst from Example 1 are stirred in 2 ml of aniline under an argon atmosphere at 170° C. for 12 h. The GC analysis of the crude product indicates the selective formation of the monoarylated compound; triarylamine is not detected. After workup by column chromatography (hexane), 680 mg (60%) of product are obtained.

GC-MS/EI: 169 (M)

### Example 26

### Coupling of iodobenzene with aniline (catalyst from Example 1)

1.37 g (6.7 mmol) of iodobenzene, 1.4 g (10.1 mmol) of caesium carbonate and 500 mg (0.7 mmol) of the catalyst from Example 1 are stirred in 2 ml of aniline under an argon atmosphere at 170° C. for 12 h. The GC analysis of the crude product indicates the selective formation of the monoarylated compound and complete conversion; triarylamine is not detected.

### Example 27

### Coupling of p-chloronitrobenzene with aniline (catalyst from Example 1)

1.06 g (6.7 mmol) of p-chloronitrobenzene, 1.4 g (10.1 mmol) of caesium carbonate and 500 mg (0.7 mmol) of the catalyst from Example 1 are stirred in 2 ml of aniline under an argon atmosphere at 170° C. for 12 h. The GC analysis of the crude product indicates the selective formation of the monoarylated compound and 83% conversion; triarylamine is not detected. After workup by column chromatography, 1.07 g (75%) of product are obtained.

GC-MS/EI: 214 (M)

### Example 28

### Coupling of bromobenzene with p-nitroaniline (catalyst from Example 1)

1.05 g (6.7 mmol) of bromobenzene, 1.4 g (10.1 mmol) of caesium carbonate and 500 mg (0.7 mmol) of the catalyst from Example 1 are mixed with 3 g of nitroaniline and stirred under an argon atmosphere at 170° C. for 12 h. The GC analysis of the crude product indicates the selective formation of the monoarylated compound and complete conversion; triarylamine is not detected.

### Example 29

### Coupling of iodobenzene with p-nitroaniline (catalyst from Example 1)

1.37 g (6.7 mmol) of iodobenzene, 1.4 g (10.1 mmol) of caesium carbonate and 500 mg (0.7 mmol) of the catalyst from Example 1 are mixed with 3 g of nitroaniline and stirred under an argon atmosphere at 170° C. for 12 h. The GC analysis of the crude product indicates the selective formation of the monoarylated compound (comparison with GC-MS/EI of 1101-4) and complete conversion; triarylamine is not detected.

### Example 30

### Coupling of iodobenzene with p-isopropylaniline (catalyst from Example 1)

1.37 g (6.7 mmol) of iodobenzene, 1.4 g (10.1 mmol) of caesium carbonate and 500 mg (0.7 mmol) of the catalyst from Example 1 are stirred in 2 ml of p-isopropylaniline under an argon atmosphere at 170° C. for 12 h. The GC analysis of the crude product indicates the formation of monoarylated and diarylated compound in a ratio of 1:1 and 70% conversion.

### Example 31

### Coupling of bromobenzene with aniline (catalyst from Example 2)

1.05 g (6.7 mmol) of bromobenzene, 1.4 g (10.1 mmol) of caesium carbonate and 490 mg (0.7 mmol) of the catalyst from Example 2 are stirred in 2 ml of aniline under an argon atmosphere at 170° C. for 12 h. The GC analysis of the crude product indicates the selective formation of the monoarylated compound and 33% conversion; triarylamine is not detected.

### Example 32

### Coupling of iodobenzene with aniline (catalyst from Example 2)

1.37 g (6.7 mmol) of iodobenzene, 1.4 g (10.1 mmol) of caesium carbonate and 490 mg (0.7 mmol) of the catalyst from Example 2 are stirred in 2 ml of aniline under an argon atmosphere at 170° C. for 12 h. The GC analysis of the crude product indicates the selective formation of the monoarylated compound and 89% conversion; triarylamine is not detected.

### Example 33

### Coupling of p-chloronitrobenzene with aniline (catalyst from Example 2)

1.06 g (6.7 mmol) of p-chloronitrobenzene, 1.4 g (10.1 mmol) of caesium carbonate and 490 mg (0.7 mmol) of the catalyst from Example 2 are stirred in 2 ml of aniline under an argon atmosphere at 170° C. for 12 h. The GC analysis of the crude product indicates the selective formation of the monoarylated compound and 63% conversion; triarylamine is not detected.

### Example 34

### Coupling of iodobenzene with aniline (catalyst from Example 3)

1.37 g (6.7 mmol) of iodobenzene, 1.4 g (10.1 mmol) of caesium carbonate and 380 mg (0.7 mmol) of the catalyst from Example 3 are stirred in 2 ml of aniline under an argon atmosphere at 170° C. for 12 h. The GC analysis of the crude product indicates the selective formation of the monoarylated compound and complete conversion; triarylamine is not detected.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. Copper complexes containing ligands of the formula (I) 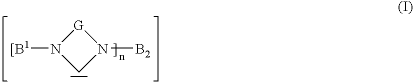
in which
G is a 1,2-ethanediyl or 1,2-ethenediyl radical which is optionally mono- or polysubstituted and
B¹ is C₅-C₁₈-aryl, C₁-C₁₈-alkyl which optionally has one or more heteroatoms from the group of oxygen, nitrogen or sulphur, or C₆-C₁₉-aralkyl and
B² is an n-valent radical having a total of 2 to 40 carbon atoms and
n is 1, 2 or 3.

2. Copper complexes according to claim 1, characterized in that G is a 1,2-ethanediyl or 1,2-ethenediyl radical which is optionally mono- or polysubstituted by C₁-C₈-alkyl.

3. Copper complexes according to claim 1, characterized in that B¹, in the case that n=1, is C₅-C₁₈-heteroaryl or C₁-C₁₈-alkyl, each of which contains one or more heteroatoms from the group of oxygen, nitrogen or sulphur, and, in the case that n=2 or 3, is C₁-C₁₈-alkyl, C₆-C₁₁-aralkyl or C₆-C₁₀-aryl.

4. Copper complexes according to claim 1, characterized in that they are of the formula (Ia) 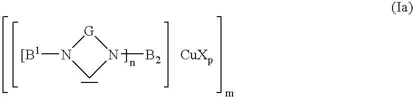
in which
n, G, B¹ und B² have the definitions specified in claim 1 and
X is halide, (C₁-C₈-haloalkyl)carboxylate, (C₁-C₈-alkyl)carboxylate, (C₁-C₈-haloalkyl)sulphonate, (C₅-C₁₈-aryl)sulphonate, cyanide, optionally fluorinated acetylacetonate, nitrate, oxinate, phosphate, carbonate, hexafluorophosphate, tetraphenylborate, tetrakis(pentafluorophenyl)borate or tetrafluoroborate and
p is 0, 1 or 2 and
m is 1, 2, 3, 4, 5 or 6.

5. Copper compexes containing ligands of formula (I) according to claim 1 selected from the group consisting of [(N,N-Dipyridyl-imidazolylidene)copper dibromide], [2,6-{bis-N-(N-methylimidazolylidene)-methyl)pyridine}copper dibromide] and [1,3-{bis-N-(N-methylimidazolylidene)methyl)-5-methylbenzene}copper dibromide].

6. A process for forming carbon-nitrogen, carbon-oxygen and carbon-sulphur bonds, and for preparing alkines comprising conducting the formation or preparation in the presence of copper complexes according to claim 1.

7. Catalysts comprising copper complexes according to claim 1.

8. Process for preparing compounds of the formula (IV),
Ar—(F—R²)ₙ (IV)
in which
n is 1, 2 or 3 and
Ar is a substituted or unsubstituted aromatic radical and
F is oxygen, sulphur, NR³, NR³CO or ethinediyl, where R³ is hydrogen, C₁-C₁₂-alkyl, C₅-C₁₈-aryl or C₆-C₁₉-arylalkyl and
R² is Ar, C₁-C₁₂-alkyl, C₁-C₁₂-haloalkyl, C₂-C₁₂-alkenyl or C₆-C₁₉-arylalkyl,
comprising reacting compounds of the formula (V)
Ar—Z (V)
in which
Ar is as defined above and
Z is chlorine, bromine, iodine, a diazonium salt or a sulphonate
with compounds of the formula (VI)
H—F—R² (VI)
in which F and R² are each as defined above and
in the presence of base and copper complexes containing ligands according to claim 1.

9. Process according to claim 8, characterized in that Ar is a carbocyclic aromatic radical having 6 to 24 framework carbon atoms or a heteroaromatic radical having 5 to 24 framework atoms, of which no, one, two or three framework atoms per cycle, but at least one framework atom in the entire molecule, are heteroatoms which are selected from the group of nitrogen, sulphur or oxygen, and the carbocyclic aromatic radicals or the heteroaromatic radicals which are optionally substituted by up to five identical or different substituents per cycle which are selected from the group of hydroxyl, chlorine, fluorine, nitro, cyano, free or protected formyl, C₁-C₁₂-alkyl, C₅-C₁₄-aryl, C₆-C₁₅-arylalkyl, —PO—[(C₁-C₈)alkyl]₂, —PO—[(C₅-C₁₄)aryl]₂, —PO—[(C₁-C₈)alkyl)(C₅-C₁₄)aryl)], tri(C₁-C₈-alkyl)siloxyl and radicals of the formula (VIIa-f),

| | | | |
|---|---|---|---|
| A-B-D-E | (VIIa) | A-E | (VIIb) |
| A-SO₂-E | (VIIc) | A-B-SO₂R⁴ | (VIId) |
| A-SO₃W | (VIIe) | A-COW | (VIIf) |

in which, each independently,
A is absent or is a C₁-C₈-alkylene radical and
B is absent or is oxygen, sulphur or NR⁴,
where R⁴ is hydrogen, C₁-C₈-alkyl, C₆-C₁₅-arylalkyl or C₅-C₁₄-aryl and
D is a carbonyl group and
E is R⁵, OR⁵, NHR⁶ or N(R⁶)₂,
where R⁵ is C₁-C₈-alkyl, C₆-C₁₅-arylalkyl, C₁-C₈-haloalkyl or C₅-C₁₄-aryl and
R⁶ is in each case independently C₁-C₈-alkyl, C₆-C₁₅-arylalkyl or C₅-C₁₄-aryl, or N(R⁶)₂ together is a cyclic amino radical and
W is OH, NH₂, or OM where M may be an alkali metal ion, half an equivalent of an alkaline earth metal ion, an ammonium ion or an organic ammonium ion.

10. Process according to claim 8, characterized in that the copper complexes containing ligands of the formula (I) are used in amounts of 0.02 mol % to 10 mol %, based on the compounds of the formula (IV) used.

11. Process according to claim 8, characterized in that the bases used are alkali metal and/or alkaline earth metal carbonates, hydrogencarbonates, alkoxides, phosphates, fluorides and/or hydroxides.

12. Process according to claim 8, characterized in that the bases used are pretreated by grinding and/or drying.
